(19) United States
(12) Patent Application Publication
(10) Pub. No.: US 2020/0063104 A1
(43) Pub. Date: Feb. 27, 2020

(54) EXPANSION OF HPSC-DERIVED GRANULOCYTIC AND LYMPHOID PROGENITORS WITH UM171

(71) Applicant: Wisconsin Alumni Research Foundation, Madison, WI (US)

(72) Inventors: Igor I. Slukvin, Verona, WI (US); Walatta-Tseyon Mesquitta, Madison, WI (US)

(21) Appl. No.: 16/547,907

(22) Filed: Aug. 22, 2019

Related U.S. Application Data
(60) Provisional application No. 62/721,209, filed on Aug. 22, 2018.

(51) Int. Cl. C12N 5/0789; C12N 5/0787

(52) U.S. Cl. CPC C12N 5/0647; C12N 5/0642; C12N 2501/42; C12N 2501/2315; C12N 2501/2302; C12N 2502/1171

(57) ABSTRACT

The present invention provides methods of amplifying or expanding CD34⁺CD43⁺CD45⁺CD41^{lo}CD235^{+/-} hematopoietic progenitor cells enriched in granulocytic progenitors (G-CFCs). In another embodiment, the present invention provides methods of producing CD34⁺CD7⁺CD41a⁻ lymphoid progenitor cells are enriched in NK progenitors.

### CROSS-REFERENCE TO RELATED APPLICATIONS

This application claims priority to U.S. Provisional Application No. 62/721,209 filed on Aug. 22, 2018, the contents of which are incorporated by reference in its entirety.

### STATEMENT REGARDING FEDERALLY SPONSORED RESEARCH

This invention was made with government support under HL116221, HL099773 and OD011106 awarded by the National Institutes of Health. The government has certain rights in the invention.

### BACKGROUND OF THE INVENTION

The field of the invention is related to methods of obtaining hematopoietic progenitors enriched in granulocyte progenitor or lymphoid progenitor cells.

Human pluripotent stem cell (hPSCs) have created alternative platforms for producing blood cells for transfusion, immunotherapies and transplantation¹⁻⁴. Advancing blood cell manufacturing from hPSCs and translating hPSC-based technologies to the clinic requires improvement after the scalability of blood cell production through enhancing hematopoietic differentiation of hPSCs and expansion of lineage-committed hematopoietic progenitors (HPs).

The pyrimido-indole derivative UM171 has been described as one the most potent small molecules that stimulates HSC expansion in vitro⁵. UM171 selectively expands EPCR⁺ cord blood HSCs with sustained short- and long-term repopulation potential⁶ and mobilized peripheral blood HSCs following lentiviral transduction⁷.

CD34⁺CD43⁺ HPs generated from hPSCs are composed of a mixture different types of progenitors, including lin⁻CD34⁺CD45⁻ and lin⁻CD34⁺CD45⁺ multipotential progenitors and CD235a⁺CD41a⁺CD45⁻ and CD235a⁺CD41a⁺CD45⁺ progenitors with erythro-megakaryocytic potential⁹⁻¹¹. It remains unclear whether UM171 uniformly expands the most primitive lin⁻CD34⁺CD43⁺ multipotential progenitors or selectively affects progenitors of particular cell lineage and whether it affects proliferation or programmed cell death of HPs.

### SUMMARY OF THE INVENTION

The present invention provides methods of differentiating, and/or expanding hematopoietic progenitors (HPs) derived from hPSCs in chemically defined conditions comprising a pyrimido-indole derivative, preferably UM171. In the examples below, we revealed that culture of hPSC-HPs in HSC expansion conditions (SFEM with added TPO, SCF, FLT3, IL3 and IL6) in the presence of UM171 predominantly expanded HPs with a unique CD34⁺CD41^{lo}CD45⁺ phenotype that were enriched in granulocytic progenitors (G-CFCs). In contrast, in lymphoid cultures on OP9-DLL4, in the presence of SCF, Flt-3, and IL7, UM171 selectively expanded CD34⁺CD7⁺CD41a⁻ lymphoid progenitors with NK and T cell potentials, and increased NK and T cell output up to 10-fold. The methods described herein are able to be used to facilitate development of protocols for robust granulocyte and lymphoid cell production from hPSCs, most preferably for adoptive immunotherapies.

In one aspect, the present disclosure provides a method of differentiating, amplifying and/or expanding CD34⁺CD43⁺CD45⁺CD41^{lo}CD235^{+/-} hematopoietic progenitor cells enriched in granulocytic progenitors (G-CFCs), the method comprising: culturing CD34+CD43+ hematopoietic progenitor cells in defined hematopoietic progenitor expansion medium comprising a pyrimidol[4,5-b] indole derivative for a sufficient time to expand a portion of the cultured cells into CD34⁺CD43⁺CD45⁺CD41^{lo}CD235^{+/-} hematopoietic progenitor cells enriched in granulocytic progenitors.

In another aspect, the present disclosure provides a method of producing, amplifying and/or expanding CD34⁺CD7⁺CD41a⁻ lymphoid progenitor cells, the method comprising: culturing CD34⁺CD43⁺ hematopoietic progenitor cells in expansion medium comprising a NOTCH ligand, IL-7, Flt-3, SCF and a pyrimidol[4,5-b] indole derivative for a sufficient amount of time to differentiate a portion of the cultured cells into CD34⁺CD7⁺CD41a⁻ lymphoid progenitor cells. In some aspects, the lymphoid progenitor cells are enriched in NK and T lymphoid progenitors.

In yet another aspect, the disclosure provides a method of generating and/or expanding natural killer (NK) cells, the method comprising: (a) culturing lymphoid progenitor cells obtained as described herein in NK differentiation medium comprising IL-2, IL-15 and with or without a pyrimidol[4,5-b] indole derivative for a sufficient amount of time to differentiate a portion of the cultured cells into NK cells.

In yet another aspect, the disclosure provides a method of generating and expanding natural killer (NK) cells, the method comprising: (a) culturing CD34⁺CD43⁺ hematopoietic progenitor cells in lymphoid differentiation medium comprising SCF, Flt3, IL-7, a NOTCH ligand and a pyrimidol[4,5-b] indole derivative for a sufficient amount of time to differentiate a portion of the cultured cells into CD34⁺CD7⁺CD41a⁻ lymphoid progenitor cells; and (b) culturing the CD34⁺CD7⁺CD41a⁻ lymphoid progenitor cells produced in step (a) in NK differentiation medium comprising IL-2, IL-15, a NOTCH ligand with or without a pyrimidol[4,5-b] indole derivative for a sufficient amount of time to differentiate a portion of the cultured cells into NK cells.

In yet a further aspect, the disclosure provides a method of generating T cells, the method comprising: culturing CD34⁺CD7⁺CD41a⁻ lymphoid progenitor cells obtained by the method o described herein in the presence of NOTCH ligand, in differentiation medium comprising SCF, Flt3, IL-7 with or without a pyrimidol[4,5-b] indole derivative for a sufficient time to generate a portion of the cultured cells into T cells.

The foregoing and other aspects and advantages of the invention will appear from the following description. In the description, reference is made to the accompanying drawings which form a part hereof, and in which there are shown, by way of illustration, preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. UM171 effect on expansion of CD34⁺CD43⁺ hPSC-derived HPs. (A) Schematic diagram of protocol used for expansion of HPs generated on day 9 H1. hESC differentiation in chemically defined conditions. (B) Representative dot plots show CD34 and CD43 expression following 5 and 7 days of expansion with UM171 or DMSO (control). (C) Histograms show that most of the cells in expansion cultures acquire CD45 expression. Dot plot demonstrates enhancing effect of UM171 on CD201 expression by CD34⁺ cells, (D) UM171 effect on % and absolute numbers of CD34⁺CD43⁺CD45⁺ HPs in cultures of hESC-derived CD43⁺ cells expanded for 5 and 7 days. Results are mean±SEM for 7 independent experiments (Day 5), and 6 independent experiments (Day 7). **p<0.01, ***p<0.001 (E) CFC potential of expanded cells. Results are mean±SEM for 7 independent experiments (Day 5), and 6 independent experiments (Day 7). **p<0.01, ***p<0.001. Representative images of colonies from HPs expanded with and without UM171 are shown. Image bar is 790 μM. (F) Cytospin showing morphology of granulocytes generated from UM171 expanded hematopoietic progenitors. Image bar is 50 μM. (G) Phenotype of neutrophils generated from hematopoietic progenitors expanded for 3 days with DMSO or UM171. (H) Phagocytosis of zymosan particles by neutrophils. Plots show histograms for cells incubated at 4° C. (filled gray; nonspecific binding control) and 37° C. (filled green). Percentages of FITC-positive cells at 37° C. minus nonspecific binding control at 4° C. are shown.

FIG. 2. UM171 enhances survival and proliferation of CD34⁺CD43⁺ hPSC-derived HPs. (A) Apoptosis evaluation using annexin V staining in cultures of hPSC-derived HPs expanded for 5 days. Bars are mean±SEM for 6 independent experiments. (B-D) Assessment of proliferative potential of CD34⁺ HPs expanded for 5 days with UM171 or DMSO using Ki67 staining (B), BrdU assay (C) and cell cycle analysis (D). Bars in (B-D) are mean±SEM for 3 independent experiments. *p<0.05. All dot plots show CD34⁺CD43⁺ gated cells.

FIG. 3. Flow cytometric and functional analysis of major cellular subsets amplified in HSC expansion conditions with UM171. (A) Representative dot plots show phenotype of major subsets of CD34⁺CD43⁺ cells identified in cultures after 3 days of expansion and gates used for their sorting (CD235a vs CD41a dot plots on the left). (B) Bar depicts mean±SEM percentage of each subset within the CD34⁺CD43⁺ population at day 3 (n=4) and day 5 (n=7) of expansion. (C) CFC composition for each sorted population. Results are mean±SEM for 6 independent experiments. (D) Identification and characterization of CD41a^{lo} population within CD45⁺ hematopoietic progenitors formed on day 9 of differentiation, before initiation of expansion. Dot plots show gates used for analysis. Pie graphs show relative proportions of each type of CFC within sorted populations. (GM is granulocyte-macrophage colonies, M is macrophage colony, G is granulocyte colony and E is erythroid colony). (E) Representative dot plots show phenotype of BrdU⁺ proliferating cells, after gating CD45⁺ cells from DMSO and UM171 expanded HPs. (F) Percentages of CD41a⁻, CD41a^{lo}, and CD41^{hi} subsets within BrdU⁺ proliferating cells after gating on the CD34⁺CD45⁺ population. Results are mean±SEM (n=3). **p<0.01, ***p<0.001. (G) Percentages of BrdU⁺ and BrdU⁻ cells within indicated subsets after 5 days of expansion, Results are mean±SEM (n=3). *p<0.05.

FIG. 4. Changes in gene expression following expansion of CD34⁺CD235a⁺CD41a⁺ progenitors in HSC expansion conditions. (A) Heatmap shows changes in expression of genes associated with HSC, myeloid, and erythro-megakaryocytic lineage development. (B) Heatmap shows expression of AhR targets in corresponding expansion conditions. (C,D) Heatmaps showing commonly upregulated or downregulated genes that demonstrated more than 2-fold change in cultures expanded with UM171 for 16 hrs and 5 days.

FIG. 5. Effect of UM171 on NK cell production from hPSC-derived HPs. (A) Schematic diagram of protocol used for NK cell differentiation of hPSC-derived HPs. (B) Representative flow cytometry dot plots displaying NK cell differentiation. (C,D) Percentages and fold change in absolute numbers of NK cells. Bars are mean±SEM for at least 6 independent experiments. **p<0.01, ***p<0.001. (E) Cytotoxicity assay against K562 targets. Representative dot plots and dose dependent cell lysis curves (mean±SEM for n=3) are shown. (F) Expression of perforin in unstimulated NK cells and INFγ following stimulation of NK cells with K562 or PMA.

FIG. 6. UM171 preferentially amplifies CD34⁺CD7⁺CD45⁺ NK cell progenitors. (A) Schematic diagram of experiments used to identify stage of NK development affected by UM171. (B) The effect of UM171 addition at stages 1 and 2 of NK cell differentiation. Bars are mean±SEM for 3 independent experiments. (C) Phenotypic features of lymphoid progenitors generated in lymphoid cultures in the presence of UM171 or DMSO. (D) Upper panel shows the effect of UM171 on the proportion of cells with lymphoid progenitor phenotype on day 7 of NK differentiation (stage 1). Lower panel shows fold change in absolute numbers of CD34⁺ progenitors in cultures with UM171, as compared to DMSO controls. Bars are mean±SEM for 3 independent experiments. **p<0.01, ***p<0.001. (E) Analysis of cell proliferation using BrdU assay in NK differentiation cultures, on days 5 and 11 of differentiation. (E) FAC-Sorting of cellular subsets from CD43⁺ HPs, on day 7 of lymphoid culture (stage 1), and assessment of their NK cell potential.

FIG. 7. Schematic diagram summarizing the effect of UM171 on expansion of hPSC-derived HPs in HSC and lymphoid expansion conditions.

FIG. 8. UM171 effect on expansion of CD34⁺CD43⁺ HPs derived from H9 hESCs and DF19-9-7T fibroblast-derived iPSCs. (A)-(B) Representative dot plots showing CD34 and CD43 expression following 5 days of expansion hPSC-derived HPs with UM171 or DMSO (control). Bar graphs showing UM171 effect on % and absolute numbers of CD34⁺CD43⁺CD45⁺ HPs. Results are mean±SEM for 3 independent experiments. *p<0.05. (C)-(D) Graphs showing CFC potential of expanded cells. (E)-(F) Representative dot plots showing apoptosis evaluation using annexin V staining, in cultures of hPSC-derived HPs expanded for 5 days. Dot plots show CD34⁺CD43⁺ gated cells. (G)-(H) Representative dot plots showing assessment of proliferative potential of CD34⁺CD45⁺ HPs expanded for 5 days with UM171 or DMSO, using (G-H) BrdU assay and (I,J) cell cycle analysis. (A), (C), (E), (G), (I) showing H9 hESCs and (B), (D), (F), (H), (J) showing DF19-9-7T iPSCs.

FIG. 9. Effect of UM171 on NK cell production from CD34⁺CD43⁺ HPs derived from H9 hESC and IISH2i-BM9 derived from bone marrow iPSCs. (A)-(B) Representative flow cytometry dot plots displaying NK cell differentiation. (C)-(D) Bar graphs showing fold changes in absolute numbers of NK cell subsets. Representative experiment is shown.

FIG. 10 provides the structure of UM171.

### DETAILED DESCRIPTION OF THE INVENTION

In describing the embodiments and claiming the invention, the following terminology will be used in accordance with the definitions set out below.

As used herein, “about” means within 5% of a stated concentration range or within 5% of a stated time frame.

The terms “defined culture medium,” “defined medium,” and the like, as used herein, indicate that the identity and quantity of each medium ingredient is known.

As used herein, the terms “chemically-defined culture conditions,” “fully defined, growth factor free culture conditions,” and “fully-defined conditions” indicate that the identity and quantity of each medium ingredient is known and the identity and quantity of supportive surface is known.

As used herein, “effective amount” means an amount of an agent sufficient to evoke a specified cellular effect according to the present invention.

As used herein, the term “pluripotent cell” and “pluripotent stem cell” means a cell capable of differentiating into cells of all three germ layers. Examples of pluripotent cells include embryonic stem (ES) cells and induced pluripotent stem (iPS) cells. As used herein, “iPS cells” refer to cells that are substantially genetically identical to their respective differentiated somatic cell of origin and display characteristics similar to higher potency cells, such as ES cells, as described herein. The cells can be obtained by reprogramming non-pluripotent (e.g., multipotent or somatic) cells.

As described in more detail in the Examples, methods of differentiating, expanding and/or amplifying hematopoietic progenitors (HPs) in serum- and feeder-free conditions are provided, specifically expanding and amplifying HPs with granulocytic potential or lymphoid potential, including NK and T cell potential, are provided. Culturing of CD34⁺CD45⁺HPs in HSC expansion medium in the presence of UM171 predominantly expands CD34⁺CD41^{lo}CD45⁺ HPs enriched in G-CFCs. In contrast, HPs in lymphoid cultures in the presence of a NOTCH ligand (e.g., DLL4 or DLL1) and UM171 can expand and amplify CD34⁺CD7⁺CD41a⁻ lymphoid progenitors with NK potential. UM171-mediated expansion of HPs is associated with increased proliferation and decreased apoptosis.

Although prior methods demonstrated that UM171 expands multipotential hematopoietic stem cells (HSCs) from cord blood, we found that UM171 exerts a different effects on hematopoietic progenitors generated from hPSCs and it's effect depends on culture conditions. As compare to prior methods, we were able to define conditions to amplify selectively granulocytic progenitors or NK cells.

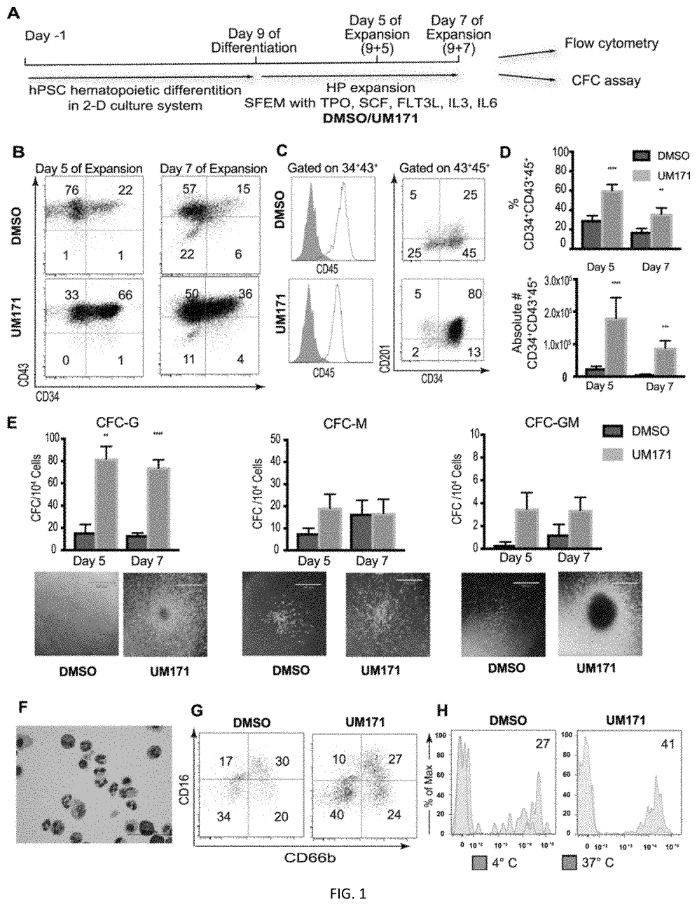

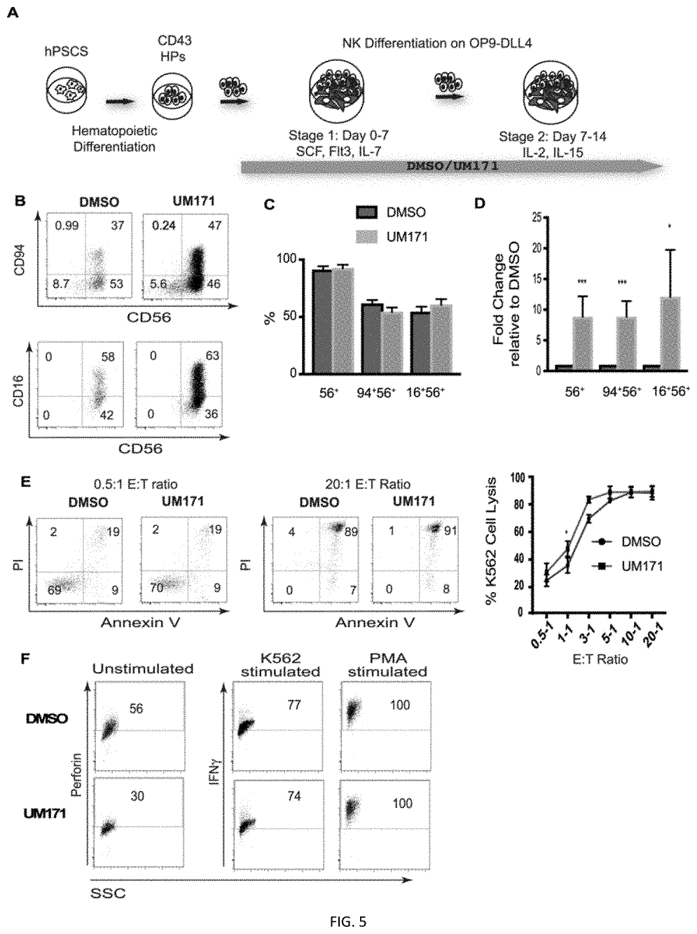

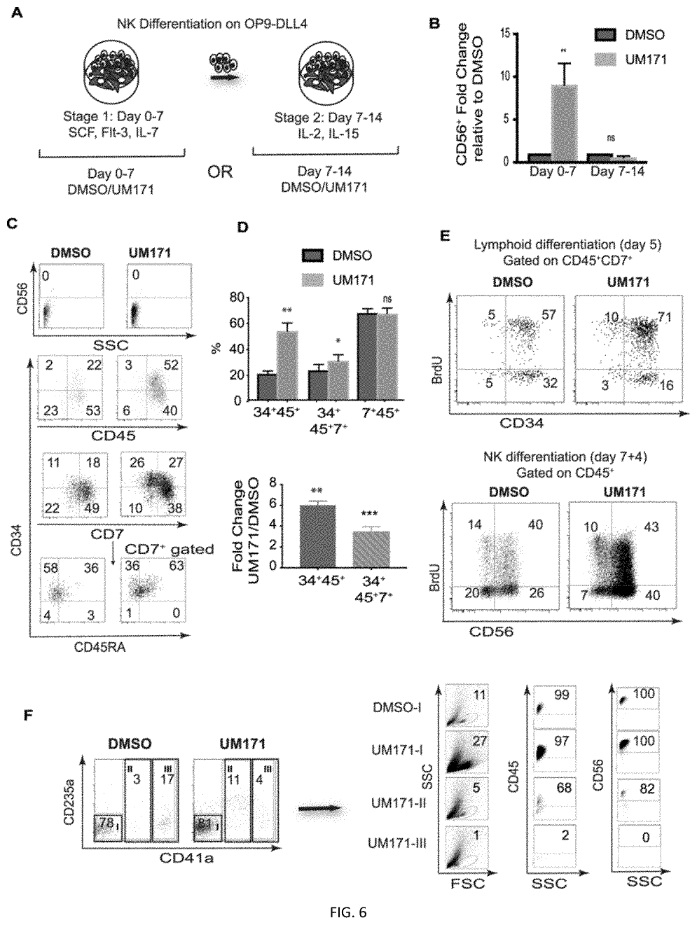

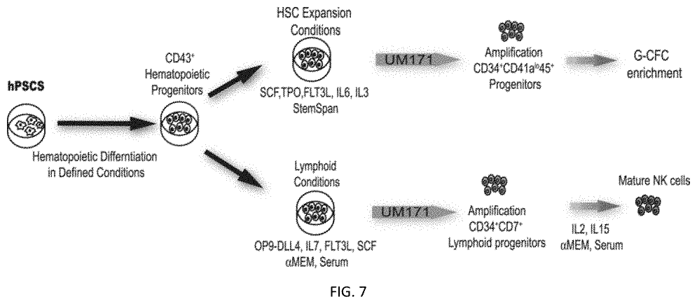

The present disclosure provides in vitro methods to generate HPs enriched in G-CFCs or HPs enriched in lymphoid progenitors with NK and T cell potential. The methods may be used to facilitate development of protocols for robust granulocyte and lymphoid cell production from hPSCs for adoptive immunotherapies. For example, as demonstrated in the Examples, the Figures provide schematic of the steps of the methods described herein. For example FIG. 1A shows an illustration demonstrating one embodiment of the method of amplifying or expanding CD34⁺CD43⁺CD45⁺CD41^{lo}CD235^{+/-} hematopoietic progenitor cells enriched in granulocytic progenitors (G-CFCs) of the present invention. FIG. 5A shows an illustration of one embodiment demonstrating a method of differentiating, amplifying and expanding CD34⁺CD7⁺CD41a⁻ lymphoid progenitor cells and NK cells from hematopoietic progenitor cells. FIG. 6A is an illustration of one embodiment demonstrating a method of differentiating, amplifying and/or expanding NK cells from hematopoietic progenitor cells (Stage 1 and 2) or lymphoid progenitor cells (Stage 2). FIG. 7 is an illustration of one embodiment demonstrating a method of differentiating, amplifying and/or expanding NK or granulocytic progenitor cells from hematopoietic progenitor or lymphoid progenitor cells.

Methods of Amplifying and Expanding CD34⁺CD41^{lo}CD45⁺ Phenotype Enriched in G-CFCs

As demonstrated in Example 1, UM171 expands hematopoietic progenitors with a unique CD34⁺CD41^{lo}CD45⁺ phenotype enriched in G-CFCs.

In one embodiment, the present disclosure provides a method of amplifying or expanding CD34⁺CD43⁺CD45⁺CD41^{lo}CD235^{+/-} hematopoietic progenitor cells enriched in granulocytic progenitors (G-CFCs), the method comprising: culturing CD34+CD43+ hemogenic progenitor cells in defined hematopoietic progenitor expansion medium comprising a pyrimidol[4,5-b] indole derivative for a sufficient time to expand a portion of the cultured cells into CD34⁺CD43⁺CD45⁺CD41^{lo}CD235^{+/-} hematopoietic progenitor cells enriched in granulocytic progenitors.

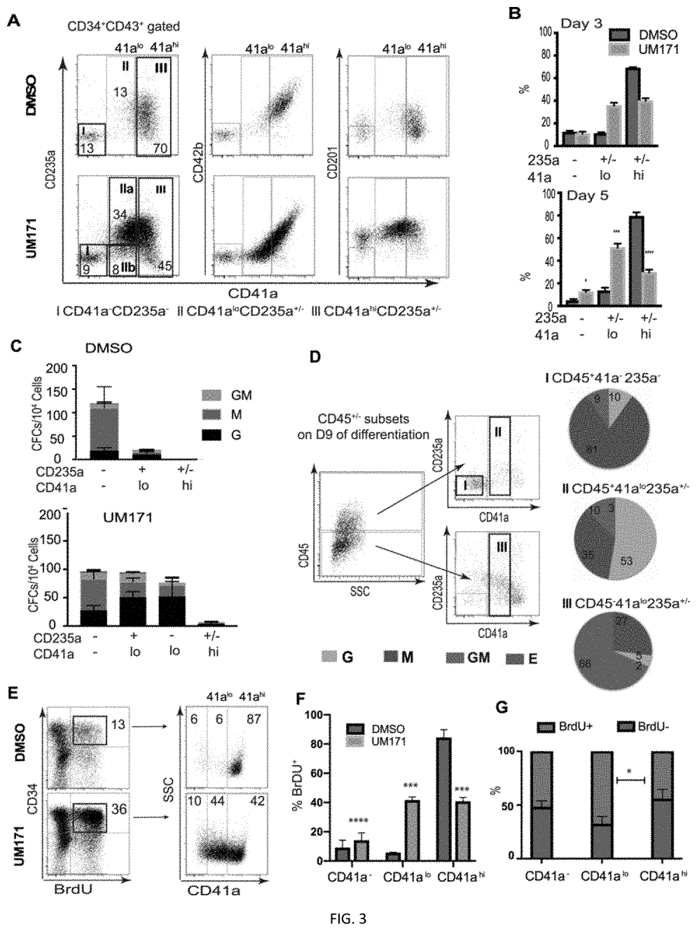

In some embodiments, the CD34⁺CD43⁺CD45⁺CD41^{lo}CD235^{+/-} hematopoietic progenitor cells enriched in granulocytic progenitors (G-CFCs) are D34⁺CD43⁺CD45⁺CD41^{lo}CD235^{+/-} hematopoietic progenitor (population II in FIG. 3).

As demonstrated in Example 1, the CD34⁺CD43⁺CD45⁺CD41^{lo}CD235^{+/-} hematopoietic progenitor cells were enriched in granulocytic progenitors (G-CFCs, see FIG. 7).

As used herein, the term “enriched” refers to a cell population in which at least 40%, preferably at least 50% of the cells have the desired characteristic. For example, CD34⁺CD43⁺CD45⁺CD41^{lo}CD235^{+/-} hematopoietic progenitor cells enriched in granulocytic progenitors (G-CFCs) refers to a population of CD34⁺CD43⁺CD45⁺CD41^{lo}CD235^{+/-} HPs in which at least 50% of the HPs are granulocyte progenitor cells.

The defined hematopoietic progenitor expansion medium used herein comprises any medium suitable for the survival in in vitro tissue culture of hematopoietic progenitor cells and allows for the growth and differentiation of the HPs to the desired cell populations. For example, a suitable medium comprises a base medium (e.g., SFEM) supplemented with TPO, SCF, Flt3, IL-3 and IL-6 and the addition of the pyrimidol[4,5-b]indole derivative. Suitable ranges of these factors are known in the art and include, for example, about 50 to about 100 ng/ml SCF; about 50 to about 100 ng/ml TPO; about 50 to about 100 ng/ml IL-6; about 5 to about 15 ng/ml IL-3 and about 5 to about 15 ng/ml Flt3. Other suitable base media that allow for the growth and proliferation of hematopoietic progenitor cells are contemplated to be able to be used in the present method. For example, suitable base media include, commercially available serum-free hematopoietic cell medium, such as, but are not limited to, StemLine™, SFEM (Serum-Free Expansion Medium, StemCell™ Technologies), StemMACS™, StemSpan™, X-Vivo™ (Lonza) among others.

In one embodiment, a sufficient time to expand a portion of the cultured cells into CD34⁺CD43⁺CD45⁺CD41^{lo}CD235^{+/-} hematopoietic progenitor cells enriched in granulocytic progenitors is at least about 3 days, alternatively at least about 5 days, alternatively at least about 7 days.

The methods contemplate the use of a pyrimidol[4,5-b] indole derivative. Suitable pyrimidol[4,5-b] indole derivatives for use in the present invention are known in the art, and can be found in, for example, U.S. Pat. No. 9,409,906, the contents of which are incorporated by reference in their entireties. Suitable pyrimidol[4,5-b] indole derivatives include, but are not limited to, for example, UM125729, UM118428, UM121184 (2-hydroxyxanthone), UM1211179, UM125454, UM232064, UM117304, and UM171 (StemCell™ Technologies).

In one embodiment, the pyrimidol[4,5-b] indole derivative is UM171. Suitable amounts of UM171 for use in the present methods are about 10 to about 100 nM, preferably from about 30 to about 100 nM, alternatively about 35 nM to about 100 nM.

In some embodiments, the method further comprises: culturing the hematopoietic progenitor cells enriched in granulocytic progenitor in granulocyte differentiation medium for a suitable time to differentiate a portion of the cells into granulocytes. Suitable granulocyte differentiation medium and conditions are known in the art. Suitable medium includes medium containing granulocyte colony-stimulating factor (G-CSF). For example, medium comprising about 50 ng/mL to about 300 ng/mL, alternatively about 100 ng/mL to about 200 ng/mL G-CSF

Suitable methods to differentiate granulocytes are known in the art and include, but are not limited to, for example, methods described in Choi et al. “Generation of mature human myelomonocytic cells through expansion and differentiation of pluripotent stem cell-derived lin−CD34+CD43+CD45+ progenitors.” The Journal of Clinical Investigation. 2009; 119(9):2818-2829. doi:10.1172/JCI38591m the contents of which are incorporated by reference in its entirety.

In some embodiments, the methods comprise a step of obtaining CD34+CD43+ hemogenic progenitor cells from human pluripotent stem cells (hPSCs). In one embodiment, CD34+CD43+ hemogenic progenitor cells are produced by culturing hPSCs on a matrix substrate using a xenogen-free and serum albumin-free mixture as described in U.S. Pat. No. 9,938,499 entitled “Methods and materials for hematoendothelial differentiation of human pluripotent stem cells under defined conditions” the contents of which are incorporated by reference in its entirety. One suitable embodiment of the method is described in Example 1 under “Hematopoietic Differentiation.” The present invention contemplates other methods of obtaining CD34+CD43+ hemogenic progenitor cells may be used in the practice of the methods described herein.

The methods described herein can use human pluripotent stem cells as the starting cell population. Suitable hPSCs include embryonic stem cells (for example, but not limited to, H9 hESC) and induced pluripotent stem cells (e.g., but not limited to, DF19-9-7T fibroblast-derived iPSC lines). Suitable examples of ESCs include, but are not limited, for example, H9 hESC. Suitable examples of iPSCs include, but are not limited to, for example, DF19-9-7T.

The present invention provides methods of amplifying hematopoietic differentiation of H1 hESCs in defined feeder- and serum-free defined conditions, and subsequently cultured cells generated on day 9 of differentiation in SFEM medium supplemented with cytokines that supports expansion of HSCs (TPO, SCF, FLT3, IL3 and IL6), with UM171 (FIG. 1A) to produce CD34⁺CD43⁺CD45⁺CD41^{lo}CD235^{+/-} hematopoietic progenitor cells enriched in granulocytic progenitors (G-CFCs). As shown in FIG. 1B-1D, the percentages and absolute numbers of CD34⁺CD43⁺ HPs almost all of which also coexpressed CD45 were significantly higher in cultures with UM171 as compared to controls (DMSO). Overall, cultures with UM171 generated up to 10 fold higher numbers of CD34⁺CD43⁺CD45⁺ HPs as compared to control, for example, about 1.5×10⁵ CD34⁺CD43⁺CD45⁺, preferably about 2×10⁵ CD34⁺CD43⁺CD45⁺. Interestingly, assessment of the CFC potential revealed that UM171 had the most dramatic effect on G-CFCs (FIG. 1E). In addition, myeloid CFCs generated from UM171 expanded HPs were much larger and denser, thereby suggesting their higher potency (FIG. 1E). Granulocyte potential of expanded cells was demonstrated by culturing the CD34⁺CD43⁺CD45⁺ in the presence of G-CFC (50-300 ng/mL) to induce differentiation towards neutrophils. As shown in FIG. 1F-1H, cells generated displayed typical neutrophil morphology and phenotype, and we capable of ingesting zymosan particles.

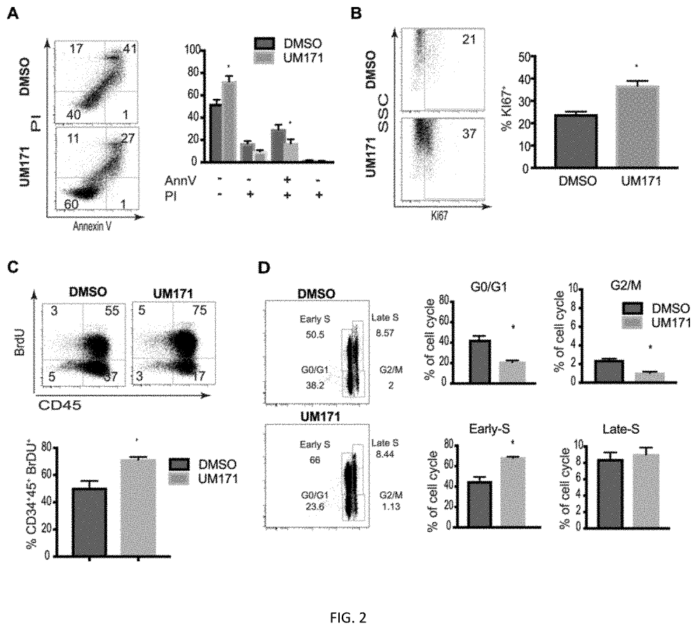

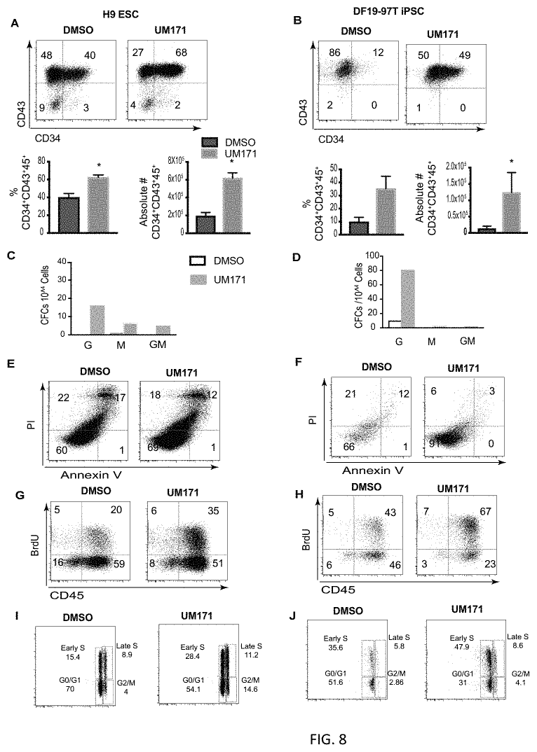

As demonstrated in the Examples, the methods described herein provided an increased number of viable CD34+CD43+ hemogenic progenitor cells and a decreased number of apoptotic, especially late apoptotic cells, in UM171 cultures as compared to controls (FIG. 2A). In addition, UM171 expansion of HPs was associated with increased proliferation, as determined by BrdU assay and Ki67 staining (FIG. 2B-C). Extending these observations, cell cycle analysis revealed that UM171 predominantly increases the proportion of HPs (CD34+CD43+ hemogenic progenitor cells) in the early S phase of the cell cycle (FIG. 2D). Similar finding are also demonstrated in FIG. 8E-J, demonstrating that CD34+CD43+ cells generated from H9 hESCs and DF19-97T hiPSCs that were expanded in HSC expansion conditions in the presence of UM171.

Our prior studies demonstrated that CD34⁺CD43⁺ HPs generated from hPSCs on day 9 of differentiation are composed of four major subsets: 1) CD235a⁺CD41a⁺CD45⁻ and 2) CD235a⁺CD41⁺CD45⁺ progenitors enriched in erythro-megakaryocytic cells (EMkPs); and 3) lin⁻CD45⁻ and 4) lin⁻CD45⁺ multipotential HPs⁹⁻¹¹. As demonstrated in Exhibit A, we found that in control conditions, CD34⁺ cells became more differentiated, acquiring CD41a^{hi}CD42b⁺ phenotype. In contrast cultures with UM171 preferentially expanded a unique CD41^{lo}235a^{+/-}CD42b⁻ population (FIG. 3A-3B). In addition, UM171 treated cultures retained HPs with CD41a⁻CD235a⁻CD34⁺ phenotype, while in control conditions this population mostly disappeared by day 5 of expansion (FIG. 3B). To determine the phenotype associated with amplified HPs, we performed sorting of three major subsets of CD34⁺CD43⁺CD45⁺ HPs from UM171 and DMSO cultures, as shown in FIG. 3A. Because CD41^{lo} population in UM171 conditions included sizable proportion of CD235a⁻ cells we additionally subdivided this population into CD235a⁺ and CD235a⁻ subsets (FIG. 3A). Assessment CFC potential of sorted subsets revealed that in UM171 cultures, most CFCs were associated with the CD41^{lo}235a^{+/-}CD42b⁻ and CD41a⁻CD235a⁻ phenotypes within CD34⁺CD43⁺CD45⁺ cells, while the CD41a^{hi}CD42b⁺ population was mostly devoid of myeloid CFCs. Interestingly, CFC numbers, especially G-CFCs were higher in CD41^{lo}235a^{+/-}CD42b⁻ population (FIG. 3C). Separation of CD41a^{lo} cells into CD235a⁺ and CD235a⁻ subsets did not reveal substantial differences in myeloid CFCs between these populations (FIG. 3C). Thus, methods of culturing HPs with UM171 in cultures with HSC expansion cytokines predominantly promote enrichment of a progenitor population with CD34⁺CD41a^{lo}CD45⁺ phenotype and enriched in unipotential granulocytic progenitors, G-CFCs (>50% of G-CFCs).

Methods of Enhancing Lymphoid Cell Production from hPSC-Derived HPs Through Expansion of CD34⁺CD7⁺CD41a⁻ NK Cell and T Cell Progenitors

In one embodiment, the disclosure provides a method of producing CD34⁺CD7⁺CD41a⁻ lymphoid progenitor cells, the method comprising: culturing CD34⁺CD43⁺ hematopoietic progenitor cells in expansion medium comprising a NOTCH ligand, IL-7, Flt-3, SCF and a pyrimidol[4,5-b] indole derivative for a sufficient amount of time to differentiate a portion of the cultured cells into CD34⁺CD7⁺CD41a⁻ lymphoid progenitor cells. In a preferred embodiment, the CD34⁺CD7⁺CD41a⁻ lymphoid progenitor cells are enriched in NK and T lymphoid progenitors.

As described above, the term “enriched in NK and T lymphoid progenitors” refers to a cell population in which at least 40%, preferably at least 50%, of the cells have the ability to form NK or T lymphoid progenitors.

Suitable NOTCH ligands for use in the present invention include, but are not limited to, for example, DLL4, DLL1-Fc, DLL1-expressing feeder cells (e.g. DLL1-expressing stroma (e.g., OP9 cells), DLL4-expressing feeder cells (e.g. DLL4-expressing OP9 cells), plates coated with DLL4-Fc, and plates coated with DLL1-Fc. In one preferred embodiment, the NOTCH ligand is DLL-4. In another embodiment, the NOTCH ligand is DLL1. Suitable amounts of NOTCH ligand would be understood by one skilled in the art. For example, plates coated with DLL4-Fc or DLL1-Fc can use a coating solution of about 2 to about 8 mg/ml.

Suitable lymphoid expansion medium includes any medium able to sustain growth and proliferation of hematopoietic progenitor cells and lymphoid progenitor cells in culture. Suitable amounts of the factors included in the expansion medium include, but are not limited to, for example, about 20-100 ng/ml SCF, about 50 to about 150 ng/ml Flt3, and about 3-10 ng/mL IL-7.

In a preferred embodiment, the pyrimidol[4,5-b] indole derivative is UM171.

In one embodiment, at least 95% of the cultured cells are CD34⁺CD7⁺CD41a⁻ lymphoid progenitor cells.

In another embodiment, the disclosure provides a method of differentiating natural killer (NK) cells, the method comprising: (a) culturing lymphoid progenitor cells in NK differentiation medium comprising IL-2, IL-15 with or without a pyrimidol[4,5-b] indole derivative for a sufficient amount of time to differentiate a portion of the cultured cells into NK cells. Suitable amounts of these factors are known in the art but include, but are not limited to, for example, about 5 to about 50 ng/ml IL-2 (e.g., 10 ng/ml), about 3 to about 25 ng/ml IL-15 (e.g., about 5 ng/ml). In some embodiments, the NK differentiation medium contains the pyrimidol[4,5-b] indole derivative. In a preferred embodiment, a majority of the cultured cells are NK cells. In one embodiment, at least 95% of the cultured cells are NK cells. In a preferred embodiment, the pyrimidol[4,5-b] indole derivative is UM171. In some embodiments, the NK differentiation medium further comprises a NOTCH ligand. Suitable NOTCH ligands include, but are not limited to, DLL4, DLL1-Fc, DLL1-expressing feeder cells (e.g. DLL1-expressing OP9 cells), DLL4-expressing feeder cells (e.g. DLL4-expressing OP9 cells), plates coated with DLL4-Fc, and plates coated with DLL1-Fc. In a preferred embodiment, the NOTCH ligand is DLL-4.

In some embodiments, the sufficient amount of time to differentiate NK cells is about one week or more.

In another embodiment, the disclosure provides a method of differentiating and expanding natural killer (NK) cells, the method comprising: (a) culturing CD34⁺CD43⁺ hematopoietic progenitor cells in lymphoid expansion medium comprising SCF, Flt3, IL-7, a NOTCH ligand and a pyrimidol[4,5-b] indole derivative for a sufficient amount of time to differentiate a portion of the cultured cells into CD34⁺CD7⁺CD41a⁻ lymphoid progenitor cells; and (b) culturing the CD34⁺CD7⁺CD41a⁻ lymphoid progenitor cells produced in step (a) in NK differentiation medium comprising IL-2, IL-15, and a NOTCH ligand with or without a pyrimidol[4,5-b] indole derivative for a sufficient amount of time to differentiate a portion of the cultured cells into NK cells. In some embodiments, the NK differentiation medium contains pyrimidol[4,5-b] indole derivative. In a preferred embodiment, the pyrimidol[4,5-b] indole derivative is UM171.

The term “expansion medium” and “lymphoid expansion medium” used herein comprises any medium suitable for the survival in in vitro tissue culture of hematopoietic progenitor cells and lymphoid progenitor cells that allows for the growth and differentiation of the HPs to the desired cell populations. Suitably, the expansion medium includes the factors SCF, Flt3, IL-7 (e.g., but not limited to, for example, 20-100 ng/ml SCF, 50-150 ng/mL Flt3, and 3-10 ng/mL IL-7), and optionally a NOTCH ligand. Suitable medium are known in the art and include, but are not limited to, for example, a-MEM, RPMI1640, DMEM, X-Vivo15, among others.

“NK differentiation medium” is any medium suitable for the growth, survival and proliferation of lymphoid progenitors and NK cells (e.g., a-MEM). Suitable amounts of factors included in the NK differentiation medium include, but are not limited to, for example, about 5 to about 50 ng/ml IL-2 (e.g., 10 ng/ml), about 3 to about 25 ng/ml IL-15 (e.g., about 5 ng/ml).

Suitably, the NK cell population produced by the method is at least 95% pure. Further, the method described herein of culturing lymphoid progenitor cells in the presence of a pyrimidol[4,5-b] indole derivative (e.g., UM171) produces about 10 times more NK cells than lymphoid progenitors cultured by methods not using a pyrimidol[4,5-b] indole derivative.

The disclosure provides a two stage method of NK cell differentiation (FIG. 6A). At stage 1, HPs are cultured with SCF, Flt-3 and IL-7 to induce lymphoid commitment. At stage 2, cells are transferred to new OP9-DLL4, and cultured with IL-2 and IL-15 to induce NK differentiation (FIG. 5A). To determine the stage affected by UM171, we added UM171 during the first 7 or the final 7-14 days of NK cell differentiation cultures (FIG. 6A). As shown in FIG. 6B, UM171 exerted its effect only when added during the first 7 days, demonstrating that addition of UM171 during the initial lymphoid differentiation stage is sufficient to amplify terminal NK yields, and suggesting that UM171 may potentiate expansion of NK progenitors rather than their differentiation into mature NK cells. Phenotypic analysis of cells generated during days 0-7 of NK cultures revealed that UM171 predominantly expands HPs with CD34⁺CD7⁺ lymphoid progenitor phenotype (FIG. 6C-6D) the majority of which are CD38⁻ and coexpress CD45RA (FIG. 6E). Most of the cells in lymphoid cultures in contrast to cultures in HSC expansion conditions were lacking CD41a and CD235a, although small proportion of cells with CD41a^{lo} and CD41^{hi} phenotype was also detected (FIG. 6F).

Culture of hPSC-derived HPs in HSC expansion conditions with UM171 preferentially expands a CD34⁺CD41^{lo}CD45⁺ population that is enriched in G-CFCs. In lymphoid cultures, UM171 preferentially expanded a CD34⁺CD45⁺CD7⁺ population with NK cell potential, while having a minimal effect on expansion of the CD34⁺CD41^{lo}CD45⁺ population (FIG. 7).

The methods described herein can be used for scaling up blood cell production from hPSCs for hPSC technologies for blood transfusion, immunotherapies and transplantation. Here we established that culture of hPSC-derived HPs in HSC expansion conditions with UM171 preferentially expanded a CD34⁺CD41^{lo}CD45⁺ population that is enriched in G-CFCs. In lymphoid cultures, UM171 preferentially expanded a CD34⁺CD7⁺CD41a⁻ population with NK cell potential, while had a minimal effect on expansion of CD34⁺CD41^{lo}CD45⁺ population.

UM171 may be used in the methods described herein for expansion of granulocytic and lymphoid progenitors with NK potential from hPSCs. These findings will help to design a protocol for scalable manufacturing of blood cells and NK cells for transfusion and immunotherapies.

The present invention has been described in terms of one or more preferred embodiments, and it should be appreciated that many equivalents, alternatives, variations, and modifications, aside from those expressly stated, are possible and within the scope of the invention.

The invention will be more fully understood upon consideration of the following non-limiting examples.

In some embodiments, kits for carrying out the methods of the present disclosure are provided. For example, a kit for culturing CD34+CD43+CD45+CD41^{lo}CD235+/− hematopoietic progenitor cells enriched in granulocytic progenitors (G-CFCs) from pluripotent stem cells is provided.

Additionally, instructions on how to culture cells are provided. In some embodiments, pluripotent stem cells that can be used for the methods are also provided in the kit. Further in some embodiments, the kits may further comprise a solid substrate on which to grow the cells, the substrate may or may not be coated with a suitable material to promote cell adhesion, e.g., extracellular matrix proteins (e.g., but not limited to, Col IV, Martigel®, etc.).

The present invention has been described in terms of one or more preferred embodiments, and it should be appreciated that many equivalents, alternatives, variations, and modifications, aside from those expressly stated, are possible and within the scope of the invention.

The invention will be more fully understood upon consideration of the following non-limiting examples

### EXAMPLES

### Example 1: UM171 Expands Distinct Types of Myeloid and NK Progenitors from Human Pluripotent Stem Cells

Scaling up blood cell production from hPSCs is critical to advancing hPSC technologies for blood transfusion, immunotherapy, and transplantation. Here we explored the potential of the HSC agonist pyrimido-indole derivative UM171, to expand hematopoietic progenitors (HPs) derived from hPSCs in chemically defined conditions. We revealed that culture of hPSC-HPs in HSC expansion conditions (SFEM with added TPO, SCF, FLT3L, IL3 and IL6) in the presence of UM171 predominantly expanded HPs with a unique CD34⁺CD41^{lo}CD45⁺ phenotype that were enriched in granulocytic progenitors (G-CFCs). In contrast, in lymphoid cultures on OP9-DLL4, in the presence of SCF, FLT3L, and IL7, UM171 selectively expanded CD34⁺CD45⁺CD7⁺ lymphoid progenitors with NK cell potential, and increased NK cell output up to 10-fold. These studies should improve our understanding of the effect of UM171 on de novo generated HPs, and facilitate development of protocols for robust granulocyte and lymphoid cell production from hPSCs, for adoptive immunotherapies.

In this Example, we evaluated the effect of UM171 on expansion and hematopoietic differentiation of HPs generated from hPSCs in chemically defined serum- and feeder-free conditions¹⁰. We revealed that culture of HPs in HSC expansion medium in the presence of UM171 predominantly expands CD34⁺CD41^{lo}CD45⁺ HPs enriched in G-CFCs. In contrast, in lymphoid cultures on OP9-DLL4, UM171 preferentially expands CD34⁺CD45⁺CD7⁺ lymphoid progenitors with robust NK potential. UM171-mediated expansion of HPs was associated with increased proliferation and decreased apoptosis.

UM171 Preferentially Expands Hematopoietic Progenitors with a Unique CD34⁺CD41^{lo}CD45⁺ Phenotype Enriched in G-CFCs.

To understand the effect of UM171 on hPSC-derived HPs and provide mechanistic insight on its action, we performed hematopoietic differentiation of H1 hESCs in defined feeder- and serum-free defined conditions for 9 days to generate HPs¹⁰. We then cultured the cells in SFEM medium supplemented with cytokines that support expansion of HSCs (TPO, SCF, FLT3L, IL3 and IL6), and with UM171 or DMSO (negative control) (FIG. 1A). As shown in FIG. 1B-1D, the percentages and absolute numbers of CD34⁺CD43⁺ HPs almost all of which also co-expressed CD45 were significantly higher in cultures with UM171 as compared to controls (DMSO). Overall, cultures with UM171 generated up to 10-fold higher numbers of CD34⁺CD43⁺CD45⁺ HPs, as compared to control cultures. Because previous studies had demonstrated that UM171 induces expression of endothelial protein C receptor (EPCR, also known as CD201) in cord blood HSC expansion cultures⁶, we analyzed the expression of this receptor in hPSC-derived HPs that were expanded in HSC conditions. As shown in FIG. 1C, expansion of hPSC-derived hematopoietic cells with UM171 was also associated with induction of CD201 expression in CD34⁺CD45⁺ HPs.

Assessment of the CFC potential of expanded cells revealed that UM171 had the most dramatic effect on G-CFCs (FIG. 1E). In addition, we noted that myeloid CFCs generated from UM171 expanded HPs were much larger and denser, thereby suggesting their higher potency (FIG. 1E). The effect of UM171 on the expansion of CD34⁺CD43⁺ HPs and G-CFCs was further confirmed using other H9 hESC and DF19-9-7T fibroblast-derived iPSC lines (Supplemental FIG. 1A-D). To confirm granulocytic potential of expanded cells we cultured them with G-CSF to induce differentiation towards neutrophils. As shown in FIG. 1F-1H, cells generated in this condition displayed typical neutrophil morphology and phenotype, and were capable of ingesting zymosan particles.

Flow cytometric analysis of apoptosis by annexin V assay demonstrated an increased number of viable cells and a decreased number of apoptotic, especially late apoptotic cells (AnnexinV⁺PI⁺), in UM171 cultures as compared to controls (FIG. 2A). In addition, UM171 expansion of HPs was associated with increased proliferation, as determined by BrdU assay and Ki67 staining (FIG. 2B-C). Extending these observations, cell cycle analysis revealed that UM171 predominantly increases the proportion of HPs in the early S phase of the cell cycle (FIG. 2D). Similar findings were obtained with CD34⁺CD43⁺ cells generated from H9 hESCs and DF19-97T hiPSCs and that were expanded in HSC expansion conditions in the presence of UM171 (Supplemental FIG. 1E-1J).

Our prior studies revealed that CD34⁺CD43⁺ HPs generated from hPSCs on day 9 of differentiation are composed of four major subsets: 1) CD235a⁺CD41a⁺CD45⁻ and 2) CD235a⁺CD41⁺CD45⁺ progenitors with erythro-megakaryocytic potential; and 3) lin⁻CD45⁻ and 4) lin⁻CD45⁺ multipotential HPs⁹⁻¹¹. To determine which types of progenitors are affected by UM171, we performed flow cytometric analysis of CD34⁺CD43⁺ cells from expansion cultures. As shown in FIG. 1C, these cells also coexpressed CD45, i.e. displayed a CD34⁺CD43⁺CD45⁺ phenotype. We found that in control conditions, CD34⁺ cells become more differentiated, acquiring a CD41a^{hi}CD42b⁺ phenotype. In contrast, cultures with UM171 preferentially expanded a unique CD41a^{lo}235a^{+/-}CD42b⁻ cell population, which coexpressed CD201 (FIG. 3A-3B). In addition, UM171 treated cultures retained HPs with CD41a⁻CD235a⁻CD34⁺ phenotype, while in control conditions this population mostly disappeared by day 5 of expansion (FIG. 3B). To determine the phenotype associated with amplified HPs, we performed sorting of three major subsets of CD34⁺CD43⁺CD45⁺ HPs from UM171 and DMSO cultures, as shown in FIG. 3A. Because the CD41a^{lo} population in UM171 conditions included a sizable proportion of CD235a⁻ cells we additionally subdivided this population into CD235a⁺ and CD235a⁻ subsets (FIG. 3A). Assessment of the CFC potential of sorted subsets revealed that in UM171 cultures, most CFCs were associated with the CD41a^{lo}235a^{+/-}CD42b⁻ and CD41a⁻CD235a⁻ phenotypes within total CD34⁺CD43⁺CD45⁺ cells, while the CD41a^{hi}CD42b⁺ population was mostly devoid of myeloid CFCs (FIG. 3C). Separation of CD41a^{lo} cells into CD235a⁺ and CD235a⁻ subsets did not reveal substantial differences in myeloid CFCs between these populations. Interestingly, we found that majority of myeloid CFCs within CD41a^{lo}235a^{+/-}CD42b⁻ population were G-CFCs. Thus, we concluded that in cultures with HSC expansion cytokines, UM171 promotes development of a progenitor population with a CD34⁺CD41a^{lo}CD45⁺ phenotype enriched in unipotential granulocytic progenitors, G-CFCs.

Although CD41a is considered a megakaryocytic lineage marker, it is also expressed by the earliest embryonic HPs and HSCs in mice¹²⁻¹⁶. In hPSC differentiation cultures, the vast majority of CD41a⁺ cells coexpress CD235a, a marker associated with the erythroid lineage^{11,17,18}. CD41a⁺CD235a⁺ cells collected from hPSC-OP9 differentiation cultures possess predominantly E- and Mk-CFC potentials^{11,17}. However, the CD235a⁺CD41a⁺ population obtained in 2D serum- and feeder-free conditions contains myeloid CFCs^{10,18}, which are mostly associated with the CD235a⁺CD41a⁺CD45⁺ fraction¹⁰. Thus, it is possible that UM171 selectively amplifies a CD34⁺CD41a^{lo}CD235a^{+/-}CD45⁺ G-CFC enriched population, which is already formed during differentiation in serum- and feeder-free defined conditions. Indeed, flow cytometric analysis on day 9 of differentiation confirmed the presence of a small cell population with CD41a^{lo}CD235a^{+/-}CD45⁺ phenotype (FIG. 3D). Sorting subsets based on CD41a and CD45 expression demonstrated G-CFC enrichment in CD41^{lo}CD45⁺ fraction. In addition, cell cycle analysis revealed that almost 90% of proliferating cells in DMSO expansion cultures were CD41a^{hi}, while UM171 cultures showed approximately equal proportions of CD41a^{lo} and CD41a^{hi} cells within BrdU⁺ fraction with up to 7-fold higher frequency of proliferating CD41a^{lo} HPs, as compared to DMSO control (FIGS. 3E and 3F). Further analysis of frequency proliferating cells within each subset in UM171 conditions, demonstrated the highest frequency of proliferating cells within CD34⁺CD41a^{lo}CD45⁺ population (FIG. 3G). Altogether, these findings suggest that the CD41a^{lo}CD235a^{+/-}CD45⁺ population enriched in G-CFCs arises from hPSCs during differentiation in defined conditions, and that UM171 selectively amplifies and maintains this population rather than induces its de novo formation.

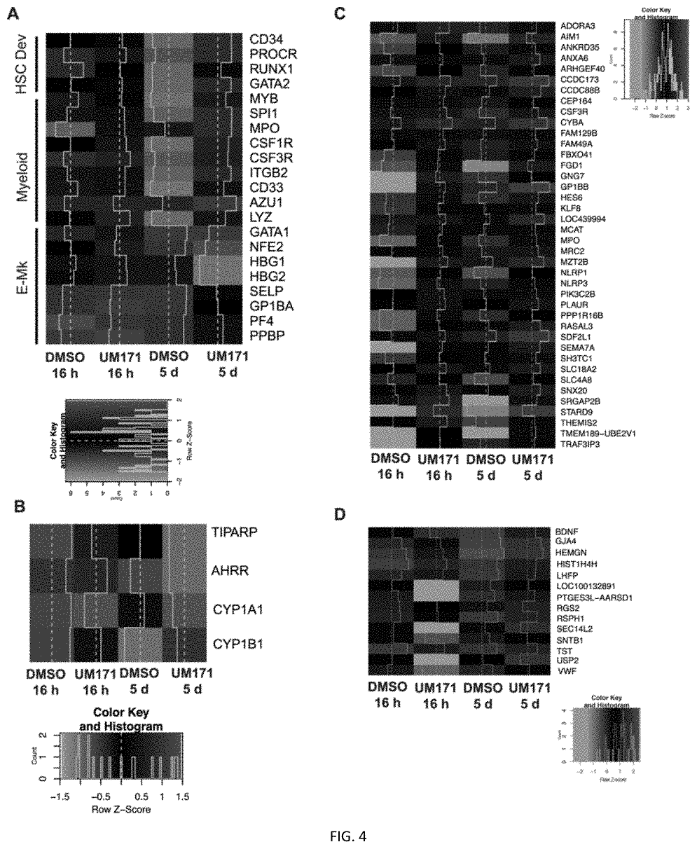

To analyze changes in molecular profile induced by UM171, we isolated CD34⁺CD235⁺CD41a⁺ cells on day 9 of differentiation, cultured them in HSC expansion conditions with UM171 or DMSO for 16 hours and 5 days, and then performed RNAseq analysis of expanded cells. Consistent with our phenotypic and functional analysis, RNAseq analysis revealed upregulation of CD34, PROCR, RUNX1 and GATA2 genes associated with HSC development along with upregulation of myeloid-lineage associated genes and down-regulation of erythro-megakaryocytic genes (FIG. 4A). Similar to previously reported findings with cord blood HSCs⁵, UM171 did not affect the expression of aryl-hydrocarbon receptor target genes, which are involved in expansion of HSCs in the presence of another potent human HSC agonist, SR1¹⁹ (FIG. 4B).

Because UM171 preferentially amplifies progenitors with G-CFC potential, the major changes in RNAseq profile may reflect enrichment of lineage-committed cells rather than downstream targets of UM171. Therefore, to better understand a direct effect of UM171 on HPs, we searched for genes that were upregulated by at least 2-fold, after 16 hours, and remained upregulated by at least 2-fold after 5 days of expansion. This analysis revealed 40 upregulated and 14 downregulated genes that were common to both, early (16 h) and late (5 days) expansion groups (FIG. 4C). The group of upregulated common genes included genes involved in granulocytic lineage development (G-CSF receptor (CSF3R) and MPO), and inflammatory response (NLRP1, NLRP3, THEMIS2 and SEMA7), suggesting that some effects of UM171 could be related to activation of inflammation associated pathways. The group of downregulated genes, included genes highly expressed in megakaryocytes (BDNF and VWF) and HEMGN gene which increases transcriptional activity of GATA1 and promotes erythroid development²⁰.

UM171 Enhances NK Production from hPSC-Derived HPs Through Expansion of CD34⁺CD7⁺ NK Cell Progenitors

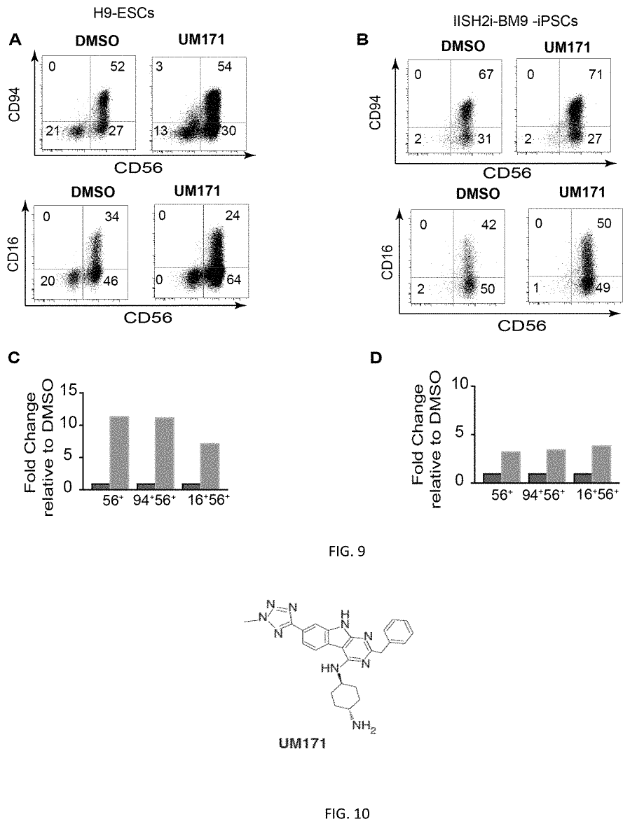

To assess the effect of UM171 on NK cell differentiation, we collected HPs from hPSC cultures on day 9 of differentiation, and cultured them on OP9-DLL4, with or without UM171, as shown in FIG. 5A. After 14 days, these cultures generated CD56⁺ NK cells that expressed CD94 and CD16, markers of mature NK cells. Although UM171 had no effect on the percentages of NK cells or their phenotype, the absolute numbers of NK cells generated with UM171 were approximately 10-fold higher as compare to DMSO controls (FIG. 5B-D). NK cells generated with UM171 possessed strong cytotoxicity, expressed perforin, and upregulated IFNγ production, following stimulation with K562 or PMA (FIG. 5E-F). These findings suggest that UM171 increases NK production through expansion, without affecting NK differentiation and function. Similar findings were obtained with HPs generated from H9 hESCs and the IISH2i-BM9 iPSC line that was derived from bone marrow mononuclear cells (FIG. 9).

Our NK cell differentiation protocol is composed of two stages (FIG. 6A). At stage 1, HPs are cultured with SCF, Flt-3 and IL-7 to induce lymphoid commitment. At stage 2, cells are transferred to new OP9-DLL4, and cultured with IL-2 and IL-15 to induce NK differentiation. To determine the stage affected by UM171, we added UM171 during the first 7 or the final 7-14 days of NK cell differentiation cultures (FIG. 6A). As shown in FIG. 6B, UM171 exerted its effect only when added during the first 7 days, demonstrating that addition of UM171 during the initial lymphoid differentiation stage is sufficient to amplify terminal NK yields, and suggesting that UM171 may potentiate expansion of NK progenitors rather than their differentiation into mature NK cells. Phenotypic analysis of cells generated during days 0-7 of NK cultures revealed that UM171 predominantly expands CD34⁺CD45⁺ HPs that are enriched in cells expressing CD7⁺ and CD45RA lymphoid progenitor markers (FIG. 6C-6D). Cell cycle analysis with BrdU demonstrated that UM171 increases the proportion of proliferating CD34⁺CD45⁺CD7⁺ progenitors while having no effect was observed in NK maturation cultures (FIG. 6E), consistent with our observation of a predominant effect of UM171 on the lymphoid progenitor stage.

Most of the cells in lymphoid cultures, in contrast to cultures in HSC expansion conditions, were lacking CD41a and CD235a, although small proportions of cells with CD41a^{lo} and CD41^{hi} phenotype were also detected (FIG. 6F). To determine whether lymphoid progenitor potential associated with CD41a⁻, CD41a^{lo}, or CD41a^{hi} fractions, we sorted these cell populations and assessed their NK cell potential. These studies revealed that the most robust NK potential resided in CD41a⁻ fraction. No NK cell potential was detected in CD41^{hi} fraction. Although the CD41a^{lo} fraction in UM171 conditions produced CD56⁺ NK cells, the efficiency of NK cell differentiation from this population was much lower (FIG. 6F). Thus, we concluded that in lymphoid conditions, UM171 expands a phenotypically and functionally different progenitor population with lymphoid potential.

Discussion

Scaling up blood cell production from hPSCs is critical for advancing hPSC technologies for blood transfusion, immunotherapies and transplantation. This can be achieved through improving the efficiency of hematopoietic differentiation from hPSCs and subsequent amplification of HPs using cytokines, growth factors and small molecules, or by overexpression of transcription factors that support self-renewal of the most primitive multipotential progenitors or lineage-restricted HPs. In the present study, we sought to explore the possibilities for enhancing blood production from hPSCs through the expansion of HPs generated from hPSCs in chemically defined conditions. Specifically, we focused on the pyrimido-indole derivative UM171, a small molecule that is known as one of the most potent enhancers of HSC expansion⁵. Here we established that culture of hPSC-derived HPs in HSC expansion conditions with UM171 preferentially expands a CD34⁺CD41^{lo}CD45⁺ population that is enriched in G-CFCs. Although we observed an increased number of CD34⁺CD41a⁻CD235a⁻CD45⁺ multipotential HPs in these conditions, these cells comprised a relatively small fraction within the entire CD34⁺ population. In lymphoid cultures, UM171 preferentially expanded a CD34⁺CD45⁺CD7⁺ population with NK cell potential, while having a minimal effect on expansion of the CD34⁺CD41^{lo}CD45⁺ population (FIG. 7).

The mechanisms of UM171 action remain unknown. It has been shown that UM171 upregulates expression of PROCR, which is involved in pathways regulating HSC retention in bone marrow²¹, and marks a highly enriched HSC population in expansion cultures⁵.

In contrast to another HSC expansion molecule SR1, which predominantly acts through inhibition of aryl hydrocarbon receptor (AHR)¹⁹, UM171 does not affect pathways associated with AHR⁵. Similar to findings with cord blood HSCs, we found that UM171 upregulates PROCR molecule in hPSC-derived HPs, but demonstrates minimal effect on AHR and AHRR expression. Although in cord blood expansion cultures, UM171 mostly decreases apoptosis without affecting proliferation of the most primitive HSCs, in hPSC-HP cultures, UM171 increased the number of cycling CD34⁺CD45⁺ cells, in addition to decreasing apoptosis.

Previous studies demonstrated that addition of UM171 or SR1 to hPSC differentiation cultures after hemogenic endothelium formation increases the number of CD34⁺CD45⁺ progenitors and CFCs^{8,22}. In addition, inhibition of AHR with SR1 promotes NK cell formation and megakaryocytic lineage development from hPSCs^{22,23}. In the present study we revealed that despite some commonalities, SR1 and UM171 differently affected hematopoiesis from hPSCs. Although both small molecules increased NK cell production from hPSCs, UM171 mostly promoted expansion of CD34⁺CD45⁺CD7⁺ lymphoid progenitors, while SR1 accelerated differentiation of already formed NK cell progenitors²². UM171 was much more potent than SR1 in expansion of NK cells, allowing us to achieve up to 10-fold increase in NK cell output, over a 14-day period, as compared to an up to 2-fold increase that was reported in prior studies with SR1, over a 28-day period²². In contrast to SR1, UM171 predominantly enhanced production of G-CFCs with CD34⁺CD41a^{lo}CD45⁺ phenotype typical of early embryonic blood progenitors.

Overall, our studies revealed UM171 to be a potent small molecule for expansion of granulocytic and NK lymphoid progenitors from hPSCs. These findings will help to design protocols for scalable manufacturing of blood cells from hPSCs for transfusion and immunotherapy.

Methods

Human Pluripotent Stem Cell and Mouse OP9-DLL4 Culture

The human ESC lines H1 (WA01) and WA09 (H9), the DF19-9-7T human fibroblast iPSC line, and the IISH2i-BM9 bone marrow-derived iPSC line were obtained from WICell Research Institute (Madison, Wis.), and maintained on vitronectin in E8 medium (WiCell). After reaching 75-80% confluence (about 3-4 days), cells were passaged by disassociation with 0.5 mM EDTA (Sigma) in PBS. E8 media was changed daily. OP9-DLL4 mouse feeder cells were maintained on 0.1% gelatin coated 10 cm dishes in OP9 growth media, consisting of α-MEM (Gibco), supplemented with 20% FBS (Hyclone). Cells were passaged at 75-80% confluency.

Hematopoietic Differentiation

Hematopoietic differentiation of hPSCs was performed using the two-dimensional hematopoietic differentiation protocol described previously¹⁰. In brief, hPSC colonies were singularized with 1× Tryple (Life Technologies) for 5 min. Singularized cells were then plated onto six-well plates coated with 0.5 μg/cm² Coll IV (Sigma Aldrich) in E8 medium, supplemented with 10 μM Rho kinase inhibitor (Tocris Y-27632). Cells were plated at a density of 10,000 to 15,000 cells/cm². After culture for 24 hours (day 0), E8 medium was changed to IF9S, supplemented with 50 ng/mL fibroblast growth factor (FGF2) (PeproTech), 50 ng/mL bone morphogenetic protein 4 (BMP4) (PeproTech), 15 ng/mL Activin A (PeproTech), and 2 mM lithium chloride (LiCl) (Sigma) for mesodermal development. TGF-β inhibitor (SB-431542, Cayman Chemicals) at 10 μM concentration was added on day 2 of differentiation. Fresh cytokine cocktails in IF9S were prepared every two days to induce stepwise differentiation through hematovascular precursors and hemogenic endothelium, into CD43 hematopoietic progenitors (day 2: 50 ng/ml FGF2 and 50 ng/ml VEGF; day 4 and 6: 50 ng/ml FGF2, VEGF, TPO, SCF, IL-6, and 10 ng/ml IL-3). Media was replaced on days 2 and 4, and added on top for day 6.

Differentiation was conducted for 9 days, first in hypoxic conditions (days 0-6), and then in normoxic conditions (days 6-9). Floating CD43 hematopoietic progenitor cells were collected at day 9, strained with a 70-μm cell strainer, and then used for experiments.

Expansion of Hematopoietic Progenitors

Hematopoietic progenitors from Day 9 differentiation cultures were expanded in StemSpan™-SFEM (Stem Cell Technologies), supplemented with 35 nM UM171 (Xcess Biosciences), or equivalent volume of DMSO, and 100 ng/ml TPO, 100 ng/ml SCF, 100 ng/ml FLT3L, 50 ng/ml IL-6, and 10 ng/ml IL-3. Expansion cultures were supplemented with fresh StemSpan™-SFEM (Stem Cell Technologies) media with the same cytokine concentrations, every 3 days. Cells were plated at up to 150,000 cells/ml. At day 5 or indicated time points, cells were collected for flow cytometric (FCM) analysis and CFU assay.

Flow Cytometry

Flow Cytometry was conducted using a MACSQuant® Analyzer 10 (Miltenyi Biotec), and the following antibodies: CD43-FITC (1G10), CD43-PerCP (1G10), CD235a-PE (HIR2), CD45-BV421 (H130), CD45-PerCP (Hl30), CD94-Fitc (HP-3D9), CD38-FITC (HIT2), CD16APC (3G8), CD7-PE (MT701), CD16-FITC (3 GB), CD42b-PE (HIP1), CD107a-FITC (H4A3) perforin-FITC (δG9), CD34 FITC (581), CD66BV421 (G10F5), CD16 PE (3G8), and CD14 PerCP (M5E2) from BD Pharmingen; CD41a-APC (REA386), CD45-APC (REA747), CD11b APC (REA713), CD201-PE (REA337), and anti-Ki67 (REA183) from Miltenyi Biotech, and CD56-PerCP (5.1H11), CD56-APC (5.1.H11), CD45RA-APC (H1100) from BioLegend. All expansions were performed using ultra-low attachment surface, tissue culture plates (CORNING).

Fluorescent Activated Cell Sorting (FACS)

Sorting was performed on a FACS Aria II cell sorter (BD). Cells of interest were harvested from differentiation cultures, and resuspended in MACS buffer (5% FBS, Gibco; 0.5 mM EDTA in PBS). The following antibodies were used: CD41a-APC, CD235a-PE, CD43-PerCP, CD45-PerCP, CD34-FITC (BD Pharmingen) and CD56-APC (BioLegend). Dead cells were counterstained with Ghost Violet 540 cell viability dye (TONBO Biosciences). Sorting gates were set with appropriate Fluorescent Minus One (FMO) controls, and only live cells were collected.

Methylcellulose Colony Forming Assay

Hematopoietic colony forming potential was assessed by combining cells of interest with 1.5 ml serum containing H4436 Methocult (Stem Cell Technologies). Cell suspensions were then transferred to 35-mm dishes and cultured for 12-14 days at 37° C. Hematopoietic colonies were scored according to cellular morphology, and CFC numbers were normalized to the number of cells plated (CFCs/10⁴) cells.

Terminal Differentiation into Mature Neutrophils

Neutrophil differentiation of DMSO or UM171 expanded hematopoietic progenitor cells was performed in IMDM (Gibco) medium, supplemented with 20% FBS (HyClone), on ultra-low attachment tissue culture plates (CORNING). Recombinant G-CSF (AMGEN) was added at a concentration of 100 ng/mL, for the first 3-4 days. Media was then topped up with an equivalent volume of fresh media, with 150-200 ng/mL G-CSF. After 7-9 days, floating cells were harvested, strained through 70-μm cell strainers, and used for flow cytometry, cytospin, and functional assays.

Phagocytosis Assay by Flow Cytometry

To analyze the phagocytic capacity of neutrophils derived from DMSO and UM171 expanded hPSC derived HPs, we incubated these neutrophils with opsonized zymosan A fluorescein particles, for 1 hour, at 37° C. or 4° C. (control). Samples were subsequently collected on ice, incubated with CD16-PE, CD66b-BV421 (BD Pharmingen), 7 and CD11b-APC (Miltenyi Biotech), and counterstained with Ghost violet 540 cell viability dye (TONBO Biosciences). Cells were then analyzed by flow cytometry.

NK Cell Differentiation

NK differentiation of hematopoietic progenitor cells was performed in α-MEM (Gibco), supplemented with 20% FBS (HyClone), on mouse OP9-DLL4 expressing feeder layer, in two stages. In stage 1, hematopoietic progenitor cells were cultured for 7 days on OP9-DLL4, with 100 ng/ml FLT3L, 40 ng/ml SCF, and 5 ng/ml IL-7 (Peprotech), and 35 nM UM171 or equivalent volume DMSO. Cells were then transferred to fresh α-MEM media, supplemented with 10 ng/ml Il-2 and 5 ng/ml IL-15 (Peprotech), and with or without UM171 treatment. Media was topped up with one volume fresh media, every 3-4 days, and cultures were transferred to new OP9-DLL4 cells, every 7 days. After 14 to 21 days, cells were harvested for flow cytometry and other analyses. OP9-DLL4 were used at 75-80% confluency.

In Vitro Cytotoxicity Assay

DMSO and UM171 differentiated hPSC-derived NK cells were purified from NK differentiation cultures by FAC-sorting with CD56-APC (BioLegend). K562 tumor cells maintained in RPMI (Gibco), supplemented with 10% FBS were stained with PK467-GFP cell membrane marker (Sigma Aldrich), according to the manufacturer's instructions. Purified CD56⁺ hPSC derived NK cells were then incubated with target K562 for 4 hours at 37° C. at effector:target (E:T) ratios of 0.5:1, 1:1, 3:1, 5:1, 10:1, and 20:1, in a final volume of 200 μl, in a 96 well plate. Following incubation, cells were collected, washed twice with MACS buffer, and then incubated with propidium iodide and annexin V-PE (BD, Biosciences) for flow based cytometric assay. Apoptosis/necrosis gates were set with appropriate Fluorescent Minus One (FMO) controls, and hPSC derived NK cells not cocultured with K562 were utilized for background subtraction.

Interferon Gamma Assays

To assess interferon gamma production, FAC-sorted NK cells were stimulated for 5 hours with PMA and ionomyocin (1:500) Cell Activation Cocktail (BioLegend), or alternately K562 tumor target cells at a 2:1 ratio. Breferidin A (1:1000; Thermo Fisher) was added at the beginning of the stimulation. NK cells that were not stimulated served as experimental controls. At the completion of incubation, all cells were washed with MACS buffer (5% FBS, Gibco; 0.5 mM EDTA in PBS), and incubated with Ghost violet 540 cell viability dye (TONBO Biosciences) and CD56-PerCP (BioLegend), for 30 minutes. Subsequently cells were permeabilized and stained with IFNγ antibodies.

Intracellular Staining for Detection of Perforin

For assessment of intracellular perforin production, NK cells were incubated with Ghost violet 540 cell viability dye (TONBO Biosciences) and CD56-PerCP (BioLegend). After washing with MACS buffer, cells were then fixed and permeabilized, following instructions in the Intracellular Fixation and Permeabilization Buffer Set (Thermo Fisher). Cells were then incubated overnight with Perforin-FITC (BD Pharmingen), and analyzed by flow cytometry.

Apoptosis and Proliferation Assays

For apoptosis detection, cells were stained with CD34-FITC, CD45-BV421, and CD43-APC (BD Pharmingen), along with PI and Annexin V-PE (BD Pharmingen), and analyzed by flow cytometry. To assess proliferation of hematopoietic progenitors, using cell proliferation antigen Ki-67, cells were incubated with CD34-PE, CD43-BV421 (BD Pharmingen), and Ghost violet 540 cell viability dye (TONBO Biosciences). Cells were then fixed and permeabilized with 70% ethanol, and stained with Ki67-APC (Miltenyi Biotech), according to the manufacturer protocol, and analyzed by flow cytometry. Cell cycle analysis of total hematopoietic progenitors was carried out using the FITC BrdU flow kit (BD Pharmingen), following manufacturer recommendations. On day four of HP expansion in HSC or lymphoid expansion conditions and on day 3 of NK differentiation cells were labelled with BrdU. After 24 hours, samples were collected and incubated with CD34-PE, CD43-BV421 (BD Pharmingen), and CD45-APC (Miltenyi Biotech); CD34-PE or 34 PerCP, CD45-BV421 (BD Pharmingen), and CD41a-APC (Miltenyi Biotech); CD45-BV421, CD7-PE, and CD34-APC (BD Pharmingen); or CD45-BV421 and CD56-APC (BD Pharmingen), and then counterstained with Ghost violet 540 cell viability dye (TONBO Biosciences). This was followed by fixation, permeabilization, and a 1 hour incubation with DNase at 37° C., to expose incorporated BrdU. After washing, cells were stained first with anti BrdU-FITC, followed by 7AAD, and flow cytometry was performed. CD34⁺CD43⁺CD45⁺ and CD34⁺CD43⁺CD45⁺CD41a⁺ hematopoietic progenitors, CD34⁺CD45⁺CD7⁺, lymphoid progenitors, or CD45⁺CD56⁺ NK cells were then analyzed for cell cycle kinetics and proliferation, through BrdU incorporation.

RNA Isolation and RNA-Seq Data Processing and Analysis.

Total RNA was isolated using RNeasy Micro Kit (Qiagen) and subjected to DNase digestion, according to the manufacturer's protocol. Isolated RNAs were used for either quantitative RT-PCR or RNA sequencing. RNA purity and integrity was evaluated by capillary electrophoresis on the Bioanalyzer 2100 (Agilent Technologies, Santa Clara, Calif.). Samples were then prepared for sequencing using the Ligation Mediated Sequencing (LM-Seq) protocol, according to the published guidelines²⁴. Final sample libraries were quantitated with the Life Technologies Qubit fluorometer and sequenced on the Illumina HiSeq 3000 (SY-401-3001). Base-calling and demultiplexing were completed with the Illumina bcl2fastq2 utility, v2.17.1.14. Following quality assessment and filtering for adapter molecules and other sequencing artifacts, the sequencing reads were aligned to transcript sequences corresponding to hg19 human genome annotation. Bowtie v 1.1.2 was used allowing two mismatches in a 25 bp seed and excluding reads with more than 200 alignments²⁵. RSEM v 1.3.0 was used to estimate isoform or gene relative expression levels in units of “transcripts per million” (tpm), as well as posterior mean estimate of the “expected counts” (the non-normalized absolute number of reads assigned by RSEM to each isoform/gene)^{26,27}. R statistical environment (R core team, 2014) was used at all stages of downstream data analysis. Count matrices were normalized using median normalization routine from EBSeq package²⁸. EBSeq with 10 iterations and pooled variance estimate was used to call for differential expression. Genes passed both Posterior Probability of Differential Expression >0.95 and fold change >2 were selected for further analysis. The accession number for the RNA-seq data reported in this paper is GEO: GSE128295 (www.ncbi.nlm.nih.gov/geo/query/acc.cgi?acc=GSE₁₂82₂₉₅).

Statistical Analysis

Statistical analysis was performed in Prism (GraphPad, San Diego, Calif.) using student t test. Data was reported as the mean±SEM of at least 3 independent experiments. Results producing p<0.05 were considered statistically significant.

### REFERENCES

1 Slukvin, I I. Generating human hematopoietic stem cells in vitro-exploring endothelial to hematopoietic transition as a portal for stemness acquisition. FEBS Lett 590, 4126-4143, doi:10.1002/1873-3468.12283 (2016).
2 Sugimura, R. et al. Haematopoietic stem and progenitor cells from human pluripotent stem cells. Nature 545, 432-438, doi:10.1038/nature22370 (2017).
3 Vo, L. T. & Daley, G. Q. De novo generation of HSCs from somatic and pluripotent stem cell sources. Blood 125, 2641-2648, doi:10.1182/blood-2014-10-570234 (2015).
4 Themeli, M. et al. Generation of tumor-targeted human T lymphocytes from induced pluripotent stem cells for cancer therapy. Nat Biotechnol 31, 928-933, doi:10.1038/nbt.2678 (2013).
5 Fares, I. et al. Cord blood expansion. Pyrimidoindole derivatives are agonists of human hematopoietic stem cell self-renewal. Science 345, 1509-1512, doi:10.1126/science.1256337 (2014).
6 Fares, I. et al. EPCR expression marks UM171-expanded CD34(+) cord blood stem cells. Blood 129, 3344-3351, doi:10.1182/blood-2016-11-750729 (2017).
7 Zonari, E. et al. Efficient Ex Vivo Engineering and Expansion of Highly Purified Human Hematopoietic Stem and Progenitor Cell Populations for Gene Therapy. Stem cell reports 8, 977-990, doi:10.1016/j.stemcr.2017.02.010 (2017).
8 Li, X. et al. Pyrimidoindole derivative UM171 enhances derivation of hematopoietic progenitor cells from human pluripotent stem cells. Stem Cell Res 21, 32-39, doi:10.1016/j.scr.2017.03.014 (2017).
9 Choi, K. D. et al. Hematopoietic and endothelial differentiation of human induced pluripotent stem cells. Stem Cells 27, 559-567, doi:10.1634/stemcells.2008-0922 (2009).
Uenishi, G. et al. Tenascin C promotes hematoendothelial development and T lymphoid commitment from human pluripotent stem cells in chemically defined conditions. Stem cell reports 3, 1073-1084, doi:10.1016/j.stemcr.2014.09.014 (2014).
11 Vodyanik, M. A., Thomson, J. A. & Slukvin, I I. Leukosialin (CD43) defines hematopoietic progenitors in human embryonic stem cell differentiation cultures. Blood 108, 2095-2105, doi:10.1182/blood-2006-02-003327 (2006).
12 Bertrand, J. Y. et al. Characterization of purified intraembryonic hematopoietic stem cells as a tool to define their site of origin. Proceedings of the National Academy of Sciences of the United States of America 102, 134-139, doi:10.1073/pnas.0402270102 (2005).
13 Emambokus, N. R. & Frampton, J. The glycoprotein IIb molecule is expressed on early murine hematopoietic progenitors and regulates their numbers in sites of hematopoiesis. Immunity 19, 33-45 (2003).
14 Ferkowicz, M. J. et al. CD41 expression defines the onset of primitive and definitive hematopoiesis in the murine embryo. Development 130, 4393-4403 (2003).
Mikkola, H. K., Fujiwara, Y., Schlaeger, T. M., Traver, D. & Orkin, S. H. Expression of CD41 marks the initiation of definitive hematopoiesis in the mouse embryo. Blood 101, 508-516, doi:10.1182/blood-2002-06-1699 (2003).
16 Mitjavila-Garcia, M. T. et al. Expression of CD41 on hematopoietic progenitors derived from embryonic hematopoietic cells. Development 129, 2003-2013 (2002).
17 Klimchenko, O. et al. A common bipotent progenitor generates the erythroid and megakaryocyte lineages in embryonic stem cell-derived primitive hematopoiesis. Blood 114, 1506-1517, doi:10.1182/blood-2008-09-178863 (2009).
18 Paluru, P. et al. The negative impact of Wnt signaling on megakaryocyte and primitive erythroid progenitors derived from human embryonic stem cells. Stem Cell Res 12, 441-451, doi:10.1016/j.scr.2013.12.003 (2014).
19 Boitano, A. E. et al. Aryl hydrocarbon receptor antagonists promote the expansion of human hematopoietic stem cells. Science 329, 1345-1348, doi:10.1126/science.1191536 (2010).
20 Zheng, W. W. et al. EDAG positively regulates erythroid differentiation and modifies GATA1 acetylation through recruiting p300. Stem Cells 32, 2278-2289, doi:10.1002/stem.1723 (2014).
21 Gur-Cohen, S. et al. PAR1 signaling regulates the retention and recruitment of EPCR-expressing bone marrow hematopoietic stem cells. Nat Med 21, 1307-1317, doi:10.1038/nm.3960 (2015).
22 Angelos, M. G. et al. Aryl hydrocarbon receptor inhibition promotes hematolymphoid development from human pluripotent stem cells. Blood 129, 3428-3439, doi:10.1182/blood-2016-07-730440 (2017).
23 Smith, B. W. et al. The aryl hydrocarbon receptor directs hematopoietic progenitor cell expansion and differentiation. Blood 122, 376-385, doi:10.1182/blood-2012-11-466722 (2013).
24 Hou, Z. et al. A cost-effective RNA sequencing protocol for large-scale gene expression studies. Sci Rep 5, 9570, doi:10.1038/srep09570 (2015).
25 Langmead, B., Trapnell, C., Pop, M. & Salzberg, S. L. Ultrafast and memory-efficient alignment of short DNA sequences to the human genome. Genome Biol 10, R25, doi:10.1186/gb-2009-10-3-r25 (2009).
26 Li, B. & Dewey, C. N. RSEM: accurate transcript quantification from RNA-Seq data with or without a reference genome. BMC Bioinformatics 12, 323, doi:10.1186/1471-2105-12-323 (2011).
27 Li, B., Ruotti, V., Stewart, R. M., Thomson, J. A. & Dewey, C. N. RNA-Seq gene expression estimation with read mapping uncertainty. Bioinformatics 26, 493-500, doi:10.1093/bioinformatics/btp692 (2010).
28 Leng, N. et al. EBSeq: an empirical Bayes hierarchical model for inference in RNA-seq experiments. Bioinformatics 29, 1035-1043, doi:10.1093/bioinformatics/btt087 (2013).

Each publication, patent, and patent publication cited in this disclosure is incorporated by reference herein in its entirety. The present invention is not intended to be limited to the foregoing examples, but encompasses all such modifications and variations as come within the scope of the appended claims.

We claim:

1. A method of producing, amplifying and/or expanding CD34⁺CD43⁺CD45⁺CD41^{lo}CD235^{+/-} hematopoietic progenitor cells enriched in granulocytic progenitors (G-CFCs), the method comprising:
a) culturing CD34+CD43+ hematopoietic progenitor cells in defined hematopoietic progenitor expansion medium comprising a pyrimidol[4,5-b] indole derivative for a sufficient time to expand a portion of the cultured cells into CD34⁺CD43⁺CD45⁺CD41^{lo}CD235^{+/-} hematopoietic progenitor cells enriched in granulocytic progenitors.

2. The method of claim 1, wherein the cultured cells are CD34⁺CD43⁺CD45⁺CD41^{lo}CD235^{+/-} hematopoietic progenitor cells enriched in granulocytic progenitors.

3. The method of claim 1, wherein at least 50% of the CD34⁺CD43⁺CD45⁺CD41^{lo}CD235^{+/-} hematopoietic progenitor cells are able to differentiate into granulocytes.

4. The method of claim 1, wherein the defined expansion medium comprises: TPO, SCF, Flt3, IL-3, and IL-6.

5. The method of claim 1, wherein the pyrimidol[4,5-b] indole derivative is UM171.

6. The method of claim 1, wherein the defined medium comprises base medium SFEM.

7. The method of claim 1, wherein the method further comprises:
(b) culturing the CD34⁺CD43⁺CD45⁺CD41^{lo}CD235^{+/-} hematopoietic progenitor cells enriched in granulocytic progenitors in granulocyte differentiation medium for a suitable time to differentiate a portion of the cells into granulocytes.

8. The method of claim 1, wherein the method comprises obtaining the CD34⁺CD43⁺ hematopoietic progenitor cells used in step (a) from human pluripotent stem cells (hPSCs) by culturing hPSCs on a matrix substrate in xenogen-free and serum albumin-free conditions.

9. A method of producing and expanding CD34⁺CD7⁺CD41a⁻ lymphoid progenitor cells, the method comprising:
culturing CD34⁺CD43⁺ hematopoietic progenitor cells in expansion medium comprising a NOTCH ligand, IL-7, Flt-3, SCF and a pyrimidol[4,5-b] indole derivative for a sufficient amount of time to differentiate and amplify a portion of the cultured cells into CD34⁺CD7⁺CD41a⁻ lymphoid progenitor cells.

10. The method of claim 9, wherein the lymphoid progenitor cells are enriched in NK and T lymphoid progenitors.

11. The method of claim 9, wherein the NOTCH ligand is selected from the group consisting of DLL4, DLL1-Fc, DLL1-expressing feeder cells (e.g. DLL1-expressing OP9 cells), DLL4-expressing feeder cells (e.g. DLL4-expressing OP9 cells), plates coated with DLL4-Fc, and plates coated with DLL1-Fc.

12. The method of claim 9, wherein the NOTCH ligand is DLL-4.

13. The method of claim 9, wherein the a pyrimidol[4,5-b] indole derivative is UM171.

14. The method of claim 9, wherein at least 95% of the cultured cells are lymphoid progenitor cells.

15. The method of claim 9, wherein the expansion medium comprises about 20-100 ng/ml SCF, about 50 to about 150 ng/ml Flt3, and about 3-10 ng/mL IL-7.

16. A method of generating and/or expanding natural killer (NK) cells, the method comprising:
culturing lymphoid progenitor cells obtained from the method of claim 9 in NK differentiation medium comprising IL-2, IL-15 and with or without a pyrimidol[4,5-b] indole derivative for a sufficient amount of time to differentiate a portion of the cultured cells into NK cells.

17. The method of claim 16, wherein the pyrimidol[4,5-b] indole derivative is UM171.

18. The method of claim 16 wherein the medium further comprises a NOTCH ligand.

19. The method of claim 18, wherein the activator of NOTCH ligand is selected from the group consisting of DLL4, DLL1-Fc, DLL1-expressing feeder cells (e.g. DLL1-expressing OP9 cells), DLL4-expressing feeder cells (e.g. DLL4-expressing OP9 cells), plates coated with DLL4-Fc, and plates coated with DLL1-Fc.

20. The method of claim 16, wherein the sufficient amount of time is about one week.

21. A method of generating and expanding natural killer (NK) cells, the method comprising:
(a) culturing CD34⁺CD43⁺ hematopoietic progenitor cells in lymphoid differentiation medium comprising SCF, Flt3, IL-7, a NOTCH ligand and a pyrimidol[4,5-b] indole derivative for a sufficient amount of time to differentiate a portion of the cultured cells into CD34⁺CD7⁺CD41a⁻ lymphoid progenitor cells; and
(b) culturing the CD34⁺CD7⁺CD41a⁻ lymphoid progenitor cells produced in step (a) in NK differentiation medium comprising IL-2, IL-15, a NOTCH ligand and a pyrimidol[4,5-b] indole derivative for a sufficient amount of time to differentiate a portion of the cultured cells into NK cells.

22. The method of claim 21, wherein the pyrimidol[4,5-b] indole derivative is UM171.

23. The method of claim 21, wherein the Notch ligand is selected from the group consisting of DLL4, DLL1-Fc, DLL1-expressing feeder cells (e.g. DLL1-expressing OP9 cells), DLL4-expressing feeder cells (e.g. DLL4-expressing OP9 cells), plates coated with DLL4-Fc, and plates coated with DLL1-Fc.

24. The method of claim 16, wherein the NK cell population produced by the method is at least 95% pure.

25. The method of claim 24, wherein about 10 times more NK cells are produced than by methods not using a pyrimidol[4,5-b] indole derivative.
